(19) United States
(12) Patent Application Publication
(10) Pub. No.: US 2026/0061165 A1
(43) Pub. Date: Mar. 5, 2026

(54) DELIVERY TOOL SYSTEM FOR ENTRY SITE TISSUE PUNCTURE

(71) Applicant: ClearPoint Neuro, Inc., Solana Beach, CA (US)

(72) Inventors: Ernesto Salegio, Solana Beach, CA (US); Jeremy Stigall, Solana Beach, CA (US); Princeton Saroha, Solana Beach, CA (US)

(21) Appl. No.: 19/319,112

(22) Filed: Sep. 4, 2025

Related U.S. Application Data
(60) Provisional application No. 63/690,430, filed on Sep. 4, 2024.

(51) Int. Cl. A61M 25/04; A61M 25/00

(52) U.S. Cl. CPC A61M 25/04; A61M 2025/0004

(57) ABSTRACT

A fluid delivery catheter includes an elongated outer sheath and an elongated inner body co-axially and slidably positioned within the outer sheath. At least one anchor is positioned on a distal region of the elongated inner body, wherein the at least one anchor transitions between an expanded state and a retracted state and wherein the at least one anchor transitions between the expanded state and the retracted state based upon relative movement between the inner body and the outer sheath. A control handle is positioned on a proximal region of the fluid delivery catheter, wherein the control handle is actuated to achieve relative movement between the inner body and the outer sheath.

### CROSS REFERENCE TO RELATED APPLICATIONS

This application claims the benefit of priority under 35 U.S. C. § 119(e) to U.S. Provisional Patent Application Ser. No. 63/690,430, filed Sep. 4, 2024. The disclosure of the application is incorporated by reference in its entirety.

### BACKGROUND

Delivery of biologics to targeted areas of the nervous system requires the localization of a delivery tool, such as a distal tip of the delivery tool, near the biological area of interest or in route to the area of interest. A flexible catheter or rigid cannula are examples form factors of such delivery tools. The delivery tool can span various lengths including just past the target tissue or extending further past the target tissue and down the vascular system. Dosing of the biologics takes place through a distal exit port of a lumen enclosed within the delivery system of choice.

A common challenge with such procedures is retaining the position of the distal exit port where the dosing can occur from significant movement within the relative space of the patient's anatomy. One key to consistent and accurate dosing is to limit any movement of the lumen and to particularly avoid complete removal of the lumen from the anatomy of interest. This desirably occurs while optimizing the waste of biologics used in a procedure.

### SUMMARY

Disclosed is a delivery system, or a delivery system component, that enables safe and secured anchoring of a delivery device inserted into the anatomy. The system uses one or more retractable and re-deployable components that enable controlled device expansion, such as laterally or horizontally in a region of an initial target location in the anatomy. The retractable and re-deployable components inhibit or stop unwanted removal of the delivery system, such as mid-procedure. They also inhibit or prevent significant movement from the internal direction that is often associated with natural body functions such as breathing.

In an embodiment, the retractable components operate pursuant to a sheathing and unsheathing structure such as in the form of coaxial sheaths that retract and release the components such as for example at a distal region of the delivery system. The system has one or more control mechanisms such as at proximal end of the system. The control mechanisms are coupled to internal mechanism that spans the usable length of the device to the location of the retractable components.

In one aspect, there is disclosed a fluid delivery catheter, comprising: an elongated outer sheath; an elongated inner body co-axially and slidably positioned within the outer sheath; at least one anchor positioned on a distal region of the elongated inner body, wherein the at least one anchor transitions between an expanded state and a retracted state and wherein the at least one anchor transitions between the expanded state and the retracted state based upon relative movement between the inner body and the outer sheath; and a control handle on a proximal region of the fluid delivery catheter, wherein the control handle is actuated to achieve relative movement between the inner body and the outer sheath.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 shows an example catheter formed of an elongated body having at least one internal lumen wherein fluid can be injected into a region of the patient via the lumen.

FIG. 2 shows an example distal tip of the catheter including anchor elements in a deployed state.

FIG. 3 shows a distal tip of the catheter including anchor elements in a retracted state.

FIG. 4 shows an example anchor element removed from the catheter.

FIG. 5 shows a distal region of the catheter wherein the anchor is formed by one or more expandable lobes.

FIG. 6 shows a distal region of the catheter wherein the anchor is formed by one or more expandable lobes.

FIG. 7 shows a distal region of the catheter wherein the anchor is formed by one or more irregular shaped wires such as in a pigtail shape such that the wires form one or more loops.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

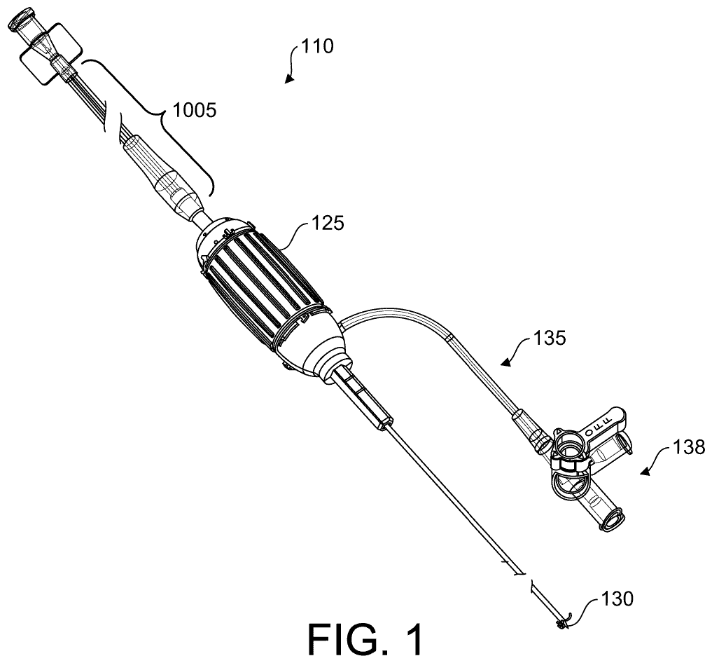

FIG. 1 shows an example catheter 110, which is formed of an elongated body having at least one internal lumen, such as an injection lumen 1005. The injection lumen spans from a proximal end of the catheter 110 to a distal exit port at a distal end of the catheter. The catheter 110 is configured to be inserted into anatomical tissue and inject fluid into a region of the patient via the lumen. The lumen 1005 can have a proximal port for entry of an injection medium and a distal port where the injection medium can leave the lumen for delivery into the patient. It should be appreciated that the catheter 110 of FIG. 1 is just an example and that the catheter 110 can vary in structure and configuration.

The catheter 110 has a catheter handle 125 or knob at a proximal region of the catheter. A distal tip of the catheter includes or forms at least one anchor element 130, as described in more detail below. In use, the catheter 110 deploys the anchor(s) 130 into a biological space, such as a cerebrospinal fluid (CSF) space, to provide distal support or anchoring relative to tissue during a procedure. As described further below, the handle 125 can be actuated to deploy and retract the anchor(s) 130. The catheter 110 can also include an aspiration/injection tube 135 configured to aspirate fluid from or inject fluid to the CSF space via a port 138.

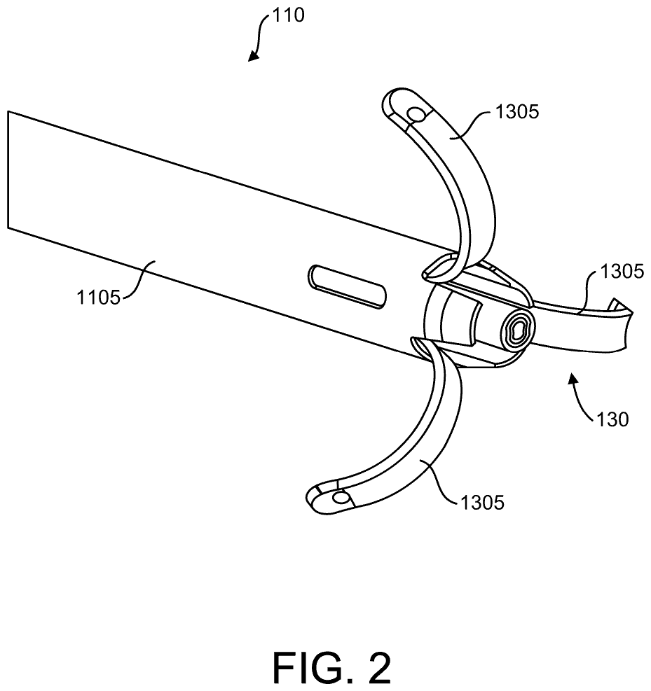

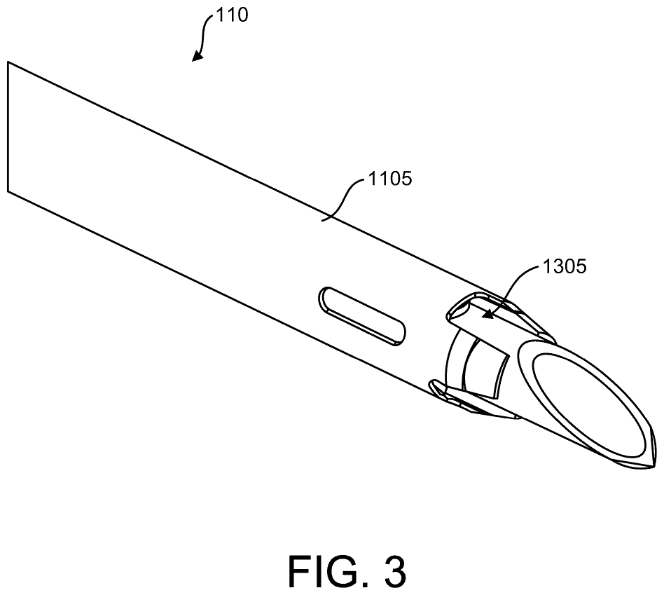

FIGS. 2 and 3 show a distal region of the catheter 110. The distal region includes an anchor element 130, which is configured to transition between an expanded or deployed state (as shown in FIG. 2) and a retracted state (as shown in FIG. 3.) An operator actuates the handle 125 to cause transition of the anchor element 130 between the two states. In the embodiment of FIGS. 2 and 3, the anchor element 130 is formed of one or more anchor elements comprised of one or more hook members that extend outward, such as laterally or horizontally outward, when deployed as shown in FIG. 2. When deployed, the hook members are shaped such that a portion initially extend laterally and distally and then curves back in a proximal direction to form a hook shape.

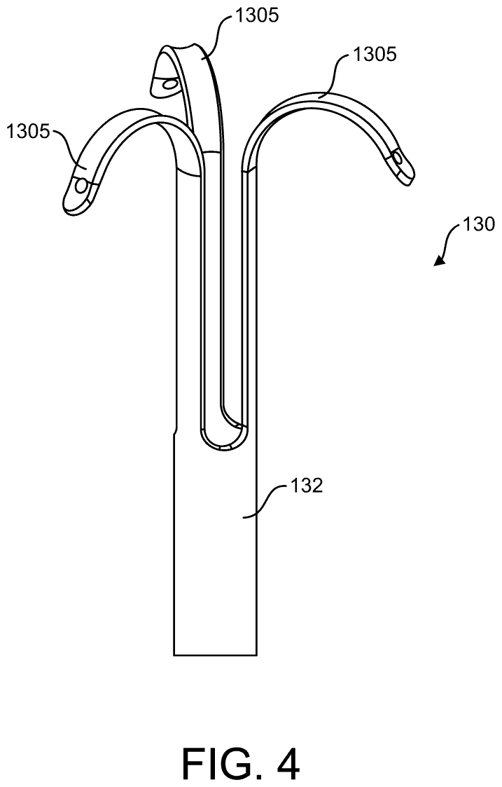

FIG. 4 shows the anchor element 130 removed from the catheter 110. The anchor element 130 is an elongated sheath or body 132 that is slidably positioned within the catheter 110 such as in a co-axial arrangement. The elongated body 132 can be an elongated, cylindrical sheath co-axially and slidably positioned within an outer cannula or sheath of the catheter 110. A distal region of the elongated body 132 forms at least one hook 1305. Each hook 1305 is constrained within an outer sheath or cannula 1105 of the catheter 110 when retracted as shown in FIG. 3. When the anchor element 130 is deployed as shown in FIG. 2, the cannula 1105 no longer constrains the anchor element 130 such that the hooks 1305 expand outward such as to engage anatomical tissue.

The anchor element 130 is deployed by co-axially sliding the elongated body 132 of the anchor element 130 relative to the outer cannula 1105 of the catheter 110. The elongated body 132 can be retractably slid to a position inside the outer cannula 1105 (as shown in FIG. 3) such that the outer cannula 1105 constrains the hooks 1305 therein. The elongated body 132 can then be slidably extended relative to the outer cannula 1105 such that the hooks 1305 are positioned distally outward of the outer cannula 1105. The hooks 1305 are biased to expand outward when unconstrained by the outer cannula 1105, as shown in FIG. 2.

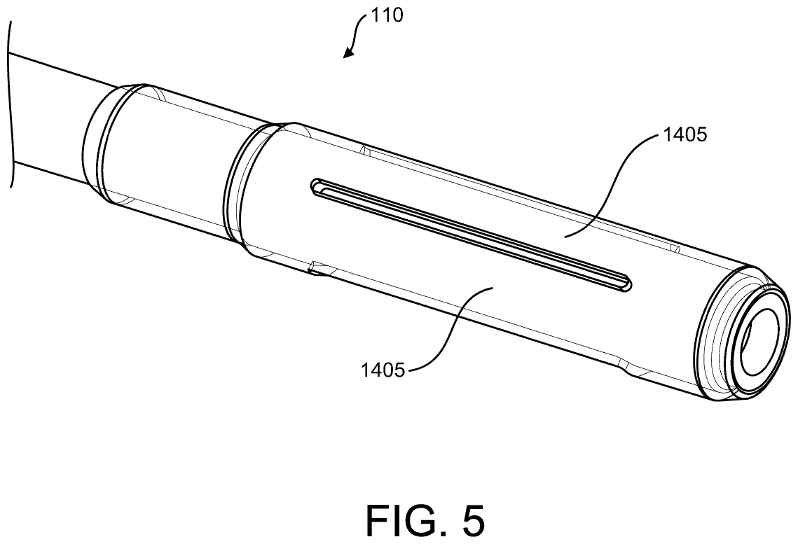

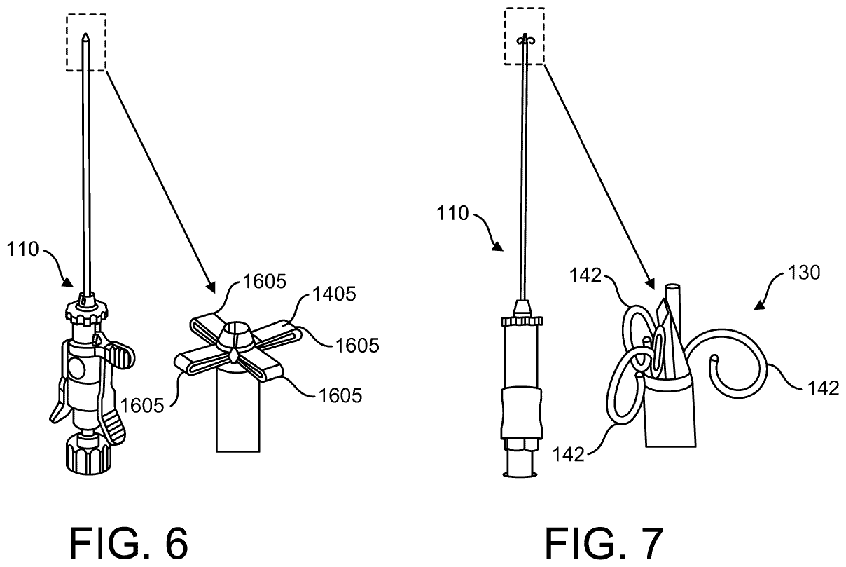

The anchor element(s) 130 can vary in structural configuration. For example, FIG. 5 shows a distal region of the catheter 110 wherein the anchor elements 130 are formed by one or more expandable jackets such as a tubular body. Each jacket has one or more lobes 1405 that are separated by slots such that the lobes 1405 that can be expanded laterally outward to a deployed state as shown in FIG. 6. The lobe can be expanded laterally outward by pulling the jacket backward (i.e., proximally) along its long axis such that a proximal region of the jacket stays fixed while a distal end of the jacket moves proximally. Such movement causes the lobe(s) 1405 to expand outward such as by bending outward at some location along the length of the lobe. When deployed, each lobe 1405 expands laterally outward and bends at a central location 1605 (or other location) to form laterally extending tab-like structures, as shown in FIG. 6.

FIG. 7 shows a distal region of the catheter 110 wherein the anchor element 130 is formed by one or more irregular shaped wires 142 such as in a pigtail shape such that the wires form one or more loops such as in a “pigtail” shape. An angular orientation of the “pigtail” wires 142 and thickness of the materials used can be in a uniform fashion or independent of one another. In the case of the pigtail loops and the hook anchors, the shape-set material can be manufactured as drawn filled tubes with materials such as platinum for improved visibility under fluoroscopy. Examples of drawn filled tubing that can be used to achieve fluoroscopic visibility of the deployed anchors are composite wire material consisting of an external nitinol tubing with an inner platinum filler. Adding fluoroscopic visibility of the anchoring features enables real time visual confirmation of proper deployment and contact of retention surfaces (horizontal expansion and the dura/tissue punctured) through computed tomography.

The quantity of anchor elements can be optimized to a desired level of internal anchoring needed for the device of interest. A combination of different shaped anchor elements can also be used to further optimize biased or directional anchoring as needed. The anchor elements can also be configured to deploy from different locations of the catheter such as to achieve a desired profiled of localized anchoring at the target tissue. For example, a first anchor can transition to an expanded state at a first location along a length of the fluid delivery catheter and a second anchor transitions to an expanded state at a second location along a length of the fluid delivery catheter, wherein the first location is offset from the second location.

For the embodiment of FIG. 5 wherein the anchor element 130 is formed by one or more expandable jackets or lobes, the anchor elements 130 can be formed of one or more outer expandable jacket or lobes 1405 such as using polymer material that can additionally be doped for fluoroscopic visibility. Doping of the polymer for fluoroscopic visibility can be accomplished prior to extrusion of the initial tubing used to form the expandable jacket or lobe. Various polymers such as PEBAX can be used as the baseline extrusion pellet material and mixed with fillers of higher fluoroscopic reactivity such as barium sulfate, bismuth subcarbonate, bismuth oxychloride, or tungsten. Fluoroscopic visibility and end jacket performance can be further tuned by adjusting the durometer of the baseline material and the ratio of that material to the radiopaque fillers being mixed in for doping. Alternative to doping the baseline material prior to the extrusion, fluoroscopic visibility can also be achieved by thermos-bonding, coating, or laminating radiopaque marker bands/layers on the distal tip of an already formed extrusion prior to lobe formation.

Lobe formation can further be achieved through a manual cutting process (e.g. blades and mechanical fixtures), or potentially the use of laser cutting. The selection of the polymer material, as well as lobe geometry, spacing, and dimension limits trauma to the tissue its anchored to when under proper use.

The anchoring features extend from a distal end or distal region of the device to the proximal end and terminate at the proximal hub/device handle. This proximal hub or the handle of the device incorporates one or more mechanisms to control the retraction and/or deployment of the anchor's deployment. Various mechanical controls can be used to enable the deployment of the anchors. Some examples are the following forms of control: a slider, a pull hub, push piston, retention clips, or rotational dials. A combination of such controls can be used together to create a simplified workflow such as by assigning a specific functional state of anchor deployment to a specific control mechanism such as a specific button. Alternatively, the controls can also be used to control the state of one anchor independent of one another or to guide sequential deployment of one anchor to the next. These controls and connections to the anchoring features are configured to allow for a delivery lumen through the center core, whether the lumen is a single, multi-lumen, or a coaxial system. Additionally, these combinations of controls may also leave enough space in their connections to allow integration with other control mechanisms with the end device (e.g. control of site puncture features) and allow for sequential operation with those features to achieve the desired workflow in a simplified manner (e.g. mechanical stops of controls dictating the critical use state of the device). Additionally, the proximal end of the handle, can include features such as Luer lock integrations per ISO standards to provide a compatible connection to a syringe or other devices.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A fluid delivery catheter, comprising:
an elongated outer sheath;
an elongated inner body co-axially and slidably positioned within the outer sheath;
at least one anchor positioned on a distal region of the elongated inner body, wherein the at least one anchor transitions between an expanded state and a retracted state and wherein the at least one anchor transitions between the expanded state and the retracted state based upon relative movement between the inner body and the outer sheath; and
a control handle on a proximal region of the fluid delivery catheter, wherein the control handle is actuated to achieve relative movement between the inner body and the outer sheath.

2. The fluid delivery catheter of claim 1, wherein the at least one anchor is a hook shaped structure that is constrained inside the outer sheath in the retracted state and expands laterally outward when unconstrained by the outer sheath.

3. The fluid delivery catheter of claim 1, wherein the at least one anchor is a pigtail shaped structure that is constrained inside the outer sheath in the retracted state and expands laterally outward when unconstrained by the outer sheath.

4. The fluid delivery catheter of claim 1, wherein the at least one anchor is a lobe that bends laterally outward at a center location of the lobe when in the expanded state.

5. The fluid delivery device of claim 1, wherein a first anchor transitions to an expanded state at a first location along a length of the fluid delivery catheter and a second anchor transitions to an expanded state at a second location along a length of the fluid delivery catheter, wherein the first location is offset from the second location.

6. The fluid delivery catheter of claim 1, wherein the control handle is actuated to cause the inner body to co-axially slide relative to the outer sheath.

7. The fluid delivery catheter of claim 5, wherein, when the at least one anchor is in the retracted state, the at least one anchor is positioned inside the outer sheath such that the outer sheath constrains the at least one anchor from expanding.

8. The fluid delivery catheter of claim 5, wherein, when the at least one anchor is in the expanded state, the at least one anchor is positioned outside the outer sheath such that least one anchor is free to expand outward.

9. The fluid delivery catheter of claim 8, wherein the at least one anchor is biased to expand outward when unconstrained.

10. The fluid delivery catheter of claim 5, wherein the control handle includes a mechanism that controls relative movement between the inner body and the outer sheath.

11. The fluid delivery catheter of claim 10, wherein the mechanism includes a slider, a pull hub, push piston, a retention clip, a rotational dial, or a combination thereof.

12. The fluid delivery catheter of claim 5, wherein the control handle can be actuated to cause a first anchor to transition to the expanded state independent of expansion of a second anchor.

13. The fluid delivery catheter of claim 5, wherein the control handle can be actuated to cause sequential expansion of a plurality of anchors.
